SEQUENCE LISTING (4 sequences)

SEQ ID NO: 1 - DNA, 20 bp, synthetic construct
  source 1..20: mol_type = other DNA
tccatgacgttcctgacgtt

SEQ ID NO: 2 - DNA, 20 bp, synthetic construct
  source 1..20: mol_type = other DNA
tccatgagcttcctgagctt

SEQ ID NO: 3 - DNA, 20 bp, synthetic construct
  source 1..20: mol_type = other DNA
acngnnganncancacaggg

SEQ ID NO: 4 - protein, 239 aa, synthetic construct
MGWSCIILFLVATATGVHSRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGGGCHHHHHH

(19) United States
(12) Patent Application Publication
(10) Pub. No.: US 2025/0360200 A1
(43) Pub. Date: Nov. 27, 2025

(54) VACCINE COMPOSITIONS AND USES THEREOF

(71) Applicant: The Regents of the University of Michigan, Ann Arbor, MI (US)

(72) Inventor: Wei Cheng, Ann Arbor, MI (US)

(21) Appl. No.: 18/866,636

(22) PCT Filed: May 30, 2023
(86) PCT No.: PCT/US2023/023846

§ 371 Date: Nov. 18, 2024

Related U.S. Application Data
(60) Provisional application No. 63/347,310, filed on May 31, 2022.

(51) Int. Cl. A61K 39/215; A61K 39/00; A61K 47/69; A61P 31/14; C12N 7/00

(52) U.S. Cl. CPC A61K 39/215; A61K 47/6911; A61P 31/14; C12N 7/00; A61K 2039/5258; A61K 2039/55555; A61K 2039/55561; A61K 2039/575; C12N 2770/20034

(57) ABSTRACT

Provided herein are vaccine compositions and uses thereof. In particular, provided herein are synthetic viral-like structures (sVLSs) based vaccines and the use of such vaccines to prevent infection by a pathogen (e.g., viral pathogen).

### STATEMENT OF RELATED APPLICATIONS

This application is a national phase application under 35 U.S.C. § 371 of PCT International Application No.: PCT/US2023/023846, filed on May 30, 2023, which claims the benefit of U.S. Provisional Patent Application No. 63/347,310, filed May 31, 2022, the entire contents of which are incorporated herein by reference for all purposes.

### STATEMENT OF GOVERNMENTAL INTEREST

This invention was made with government support under AI155653 awarded by the National Institutes of Health. The government has certain rights in the invention.

### SEQUENCE LISTING

The text of the computer readable sequence listing filed herewith, titled “UM-40942-252_SQL”, created Feb. 17, 2025, having a file size of 4,812 bytes, is hereby incorporated by reference in its entirety.

### FIELD

Provided herein are vaccine compositions and uses thereof. In particular, provided herein are synthetic viral-like structures (sVLSs) based vaccines and the use of such vaccines to prevent infection by a pathogen (e.g., viral pathogen).

### BACKGROUND

The rapid global spread of severe acute respiratory syndrome coronavirus 2 (SARS-COV-2) has had a devastating impact on human health and global economies. Global cases of SARS-CoV-2 infection have exceeded 100 million, and more than 2.1 million fatalities have occurred. Several promising vaccines, including RNA-based, adenovirus vectored, and inactivated viral vaccines are in the final phases of clinical testing and some have now received emergency use authorizations (EUAs) in various countries. Candidate subunit vaccines are soon to follow. However, many parameters remain to be determined for first generation vaccines, such as the duration and breadth of conferred immunity, whether or not vaccine induced immunity is sterilizing, and real-world efficacy, particularly in cohorts which traditionally display low response rates to vaccination, such as the elderly and immunocompromised.

In addition, new genetic variants of SARS-COV-2 have arisen which are reported to show higher transmissibility, increased virulence, and the potential for escape from current vaccines. Thus, it is clear that successful control of the pandemic will require vaccines which can provide not only robust and long-lasting protection, but also confer broad immunity towards these variants and potential future variants.

### SUMMARY

Provided herein are vaccine compositions and uses thereof. In particular, provided herein are synthetic viral-like structures (sVLSs) based vaccines and the use of such vaccines to prevent infection by a pathogen (e.g., viral pathogen).

The sVLSs described herein allow for (1) potent activation of neutralizing antibody response in both normal and antibody-deficient setting; (2) optimization of immunogenicity, neither of which is available in existing vaccine platforms. In addition, the sVLSs are stable and provide easy access to people around the world in contrast to the need of ultracold freezing conditions.

For example, in some embodiments, provided herein is synthetic viral-like structure (sVLS), comprising: a liposome comprising a polypeptide antigen covalently conjugated to the surface of the liposome via a maleimide group of a lipid in the liposome and a thiol group of the polypeptide antigen. In some embodiments, the thiol group is on a cysteine of the polypeptide antigen (e.g., via site-specifically engineering or naturally found in the polypeptide antigen). In some embodiments, the cysteine is selectively reduced (e.g., via tris(2-carboxyethyl) phosphine (TCEP)).

The sVLS allow for tuning of the density of polypeptide antigen (e.g., via concentration of lipid or polypeptide) to tune immunogenicity (e.g., for different subjects). The present disclosure is not limited to a particular density of polypeptide antigen. In some embodiments, each liposome comprises at least 10, 15, 20, 25, 30, 35, 40, 45, or 50 molecules of the polypeptide antigen.

The present disclosure is not limited to particular lipids. In some embodiments, the lipid is 1,2-distearoyl-sn-glycero-3-phosphocholine (DSPC), 1,2-distearoyl-sn-glycero-3-phosphoethanolamine-N-[maleimide (polyethylene glycol)-2000], 1,2-diheptadecanoyl-sn-glycero-3-phosphocholine, or 1,2-dinonadecanoyl-sn-glycero-3-phosphocholine.

In some embodiments, the liposome encapsulates a nucleic acid (e.g., DNA or RNA) adjuvant (e.g., comprising an all-natural phosphodiester backbone). The present disclosure is not limited to a particular nucleic acid adjuvant. Examples include but are not limited to, 5′-TCCATGACGTTCCTGACGTT-3′ (SEQ ID NO: 1), TCCATGAGCTTCCTGAGCTT-3′ (SEQ ID NO: 2), or 5′-ACUGUUGAUUCAUCACAGGG-3′ (SEQ ID NO: 3).

The present disclosure is not limited to a particular antigen. In some embodiments, the antigen is a pathogen (e.g., viral) antigen. In some specific embodiments, the polypeptide antigen is a viral (e.g., SARS-COV-2) receptor binding domain (RBD).

Also provided is a composition (e.g., pharmaceutical composition), kit, or system comprising an sVLS described herein. In some embodiments, the composition, kit, or system further comprises a delivery device and/or pharmaceutically acceptable carrier.

Further embodiments provide a method of generating an immune response to an antigen and/or protecting against infection by a pathogen, comprising, administering a composition described herein to a subject in need thereof.

Yet other embodiments provide the use of a composition described herein to generate an immune response to an antigen in a subject and/or prevent a viral infection in a subject.

Additional embodiments are described herein.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows sequence of the engineered RBD protein and reactive cysteine mapping using mass spectrometry.

FIG. 2 shows conjugation of RBD with maleimide-containing liposomes. Lane 1: molecular weight marker. Lane 2: the RBD-liposome conjugate before size exclusion chromatography (SEC). Lane 3: the admixture of RBD-Ala and liposome under the same conditions as RBD-liposome in Lane 2. Lane 4: SEC purified RBD-liposome.

FIG. 3 shows efficacy of RBD-conjugated sVLS as a function of storage time at 4° C. The efficacy was evaluated by comparing the RBD-specific IgG elicited in C57BL6/J mice on Day 5 and Day 11 upon a single subcutaneous (SC) injection of the sVLS stored at 4° C. for 2 days (Condition 1), 6 months (Condition 2) and 1 year (Condition 3) after the initial preparation.

FIG. 4 shows anti-RBD and anti-HEL IgG and IgM antibody response in mice with C57BL/6 genetic background measured 5 days and 11 days after a single injection with various agents. (a) ELISA OD values for anti-RBD IgG (circles with black edge for D5 and bars with black edge for D11) and anti-RBD IgM (circles with blue edge for D5 and bars with blue edge for D11) from 1:100 diluted mouse sera. (b) ELISA OD values for anti-HELD IgG (circles with black edge for D5 and bars with black edge for D11) and anti-HELD IgM (circles with blue edge for D5 and bars with blue edge for D11) from 1:100 diluted mouse sera.

FIG. 5 shows anti-RBD IgG antibody responses in mice with C57BL/6 genetic background measured 5 days and 11 days after a single SC injection of various agents. ELISA OD values for anti-RBD IgG (circles for D5 and bars for D11 in 5a) from 1:100 diluted mouse sera and corresponding titer values (circles for D5 and squares for D11 in 5b).

FIG. 6 shows that anti-RBD antibody elicited by RBD-conjugated sVLS can potently neutralize HIV-1 based pseudovirions that display the full-length S protein of the SARS-COV-2.

FIG. 7 shows anti-HELD IgG antibody responses in mice with C57BL/6 genetic background measured 5 days and 11 days after a single SC injection of various agents. ELISA OD values for anti-HEL IgG (circles for D5 and bars for D11 in 7a) from 1:100 diluted mouse sera and corresponding titer values (circles for D5 and squares for D11 in 7b).

FIG. 8 shows time courses of anti-RBD IgG response in both C57BL6/J and BALB/cJ mice upon a single SC injection of RBD-conjugated sVLS of varied epitope densities with and without DNA1.

FIG. 9 shows time courses of anti-HELD IgG response in both C57BL6/J and BALB/cJ mice upon a single SC injection of HELD-conjugated sVLS of varied epitope densities with and without DNA1.

### DEFINITIONS

To facilitate an understanding of the present technology, a number of terms and phrases are defined below. Additional definitions are set forth throughout the detailed description.

The terms “host” or “subject,” as used herein, refer to an individual to be treated by (e.g., administered) compositions and methods of the present disclosure. Subjects include, but are not limited to, mammals (e.g., murines, simians, equines, bovines, porcines, canines, felines, and the like), and most preferably includes humans. In the context of the disclosure, the term “subject” generally refers to an individual who will be administered (e.g., injectably and/or intranasal administered) or who has been administered one or more compositions of the present disclosure.

As used herein, the term “sample” is used in its broadest sense and encompasses materials obtained from any source. As used herein, the term “sample” is used to refer to materials obtained from a biological source, for example, obtained from animals (including humans), and encompasses any fluids, solids, and/or tissues. In particular embodiments of the present disclosure, biological samples include blood and blood products such as plasma, serum and the like. However, these examples are not to be construed as limiting the types of samples that find use with the present disclosure.

As used herein, the term “adjuvant” refers to any substance that can stimulate an immune response. Some adjuvants can cause activation of a cell of the immune system (e.g., an adjuvant can cause an immune cell to produce and secrete a cytokine). Examples of adjuvants that can cause activation of a cell of the immune system include, but are not limited to, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, an adjuvant is a nucleic acid.

As used herein, the term “immune response” and grammatical equivalents thereof refer to a response by the immune system of a subject. For example, immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the terms “toll receptors” and “TLRs” refer to a class of receptors (e.g., TLR1, TLR2, TLR3, TLR4, TLR5, TLR6, TLR7, TLR8, TLR9, TLR10, TLR 11) that recognize special patterns of pathogens, termed pathogen-associated molecular patterns (see, e.g., Janeway and Medzhitov, (2002) Annu. Rev. Immunol., 20:197-216). These receptors are expressed in innate immune cells (e.g., neutrophils, monocytes, macrophages, dendritic cells) and in other types of cells such as endothelial cells. Their ligands include bacterial products such as LPS, peptidoglycans, and lipopeptides. TLRs are receptors that bind to exogenous ligands and mediate innate immune responses leading to the elimination of invading microbes. The TLR-triggered signaling pathway leads to activation of transcription factors including NFκB, which is important for the induced expression of proinflammatory cytokines and chemokines. TLRs also interact with each other. For example, TLR2 can form functional heterodimers with TLR1 or TLR6. The TLR2/1 dimer has a different ligand binding profile than the TLR2/6 dimer (Ozinsky et al., PNAS, 97 (25): 13766-13771 (2000)). In some embodiments, an adjuvant activates cell signaling through a TLR (e.g., TLR2, TLR3, and/or TLR4). Such an adjuvant can activate TLRs (e.g., TLR2, TLR3, and/or TLR4) by, for example, interacting with TLRs (e.g., NE adjuvant binding to TLRs) or activating any downstream cellular pathway that occurs upon binding of a ligand to a TLR. NE adjuvants described herein that activate TLRs can also enhance the availability or accessibility of any endogenous or naturally occurring ligand of TLRs. A NE adjuvant that activates one or more TLRs can alter transcription of genes, increase translation of mRNA, or increase the activity of proteins that are involved in mediating TLR cellular processes. For example, NE adjuvants described herein that activate one or more TLRs (e.g., TLR2, TLR3, and/or TLR4) can induce expression of one or more cytokines (e.g., IL-8, IL-12p40, and/or IL-23).

As used herein, the term “immunity” refers to protection from disease (e.g., preventing or attenuating (e.g., suppression) of a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired/adaptive (e.g., immune responses that are mediated by B and T cells following a previous exposure to antigen (e.g., that exhibit increased specificity and reactivity to the antigen)).

As used herein, the term “antibody” refers to an immunoglobulin molecule that is typically composed of two identical pairs of polypeptide chains, each pair having one “light” (L) chain and one “heavy” (H) chain. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The constant regions of the antibodies may mediate the binding of the immunoglobulin to host tissues or factors, including various cells of the immune system (e.g., effector cells) and the first component (C1q) of the classical complement system. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of each heavy/light chain pair (VH and VL), respectively, form the antibody binding site. The term “antibody” encompasses an antibody that is part of an antibody multimer (a multimeric form of antibodies), such as dimers, trimers, or higher-order multimers of monomeric antibodies. It also encompasses an antibody that is linked or attached to, or otherwise physically or functionally associated with, a non-antibody moiety. Further, the term “antibody” is not limited by any particular method of producing the antibody. For example, it includes, inter alia, recombinant antibodies, synthetic antibodies, monoclonal antibodies, polyclonal antibodies, bi-specific antibodies, and multi-specific antibodies.

In the context of the present disclosure, a “neutralizing antibody” is an antibody that binds to a pathogen such as a virus (e.g., a coronavirus) and interferes with the virus' ability to infect a host cell.

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

As used herein, the terms “immunogen” and “antigen” are used interchangeably to refer to an agent (e.g., a microorganism (e.g., bacterium, virus, or fungus) and/or portion or component thereof (e.g., protein, glycoprotein, lipoprotein, peptide, glycopeptide, lipopeptide, toxoid, carbohydrate, tumor-specific antigen, etc.)) that is capable of eliciting an immune response in a subject. In preferred embodiments, immunogens elicit immunity against the immunogen.

By “epitope” is meant a sequence of an antigen that is recognized by an antibody or an antigen receptor. Epitopes also are referred to in the art as “antigenic determinants.” In certain embodiments, an epitope is a region of an antigen that is specifically bound by an antibody or a T cell receptor. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The immunogen or antigen can be a protein or peptide of viral, bacterial, parasitic, fungal, protozoan, prion, cellular, or extracellular origin, which provokes an immune response in a mammal, preferably leading to protective immunity. An immunogen or antigen also may be based on one or more antigenic components of a particular organism and can be generated using recombinant DNA technology.

“Nasal application”, as used herein, means applied through the nose into the nasal or sinus passages or both. The application may, for example, be done by drops, sprays, mists, coatings or mixtures thereof applied to the nasal and sinus passages.

The term “vaccine,” as used herein, refers to a biological preparation that stimulates a subject's immune system against a particular infectious agent and provides active acquired immunity to a particular infectious disease. A vaccine typically contains an agent that resembles a disease-causing microorganism and is often made from weakened or killed forms of the microbe, its toxins, or one of its surface proteins. The agent stimulates a subject's immune system to recognize the agent as a threat, destroy it, and to further recognize and destroy any of the microorganisms associated with that agent that it may encounter in the future. Vaccines can be prophylactic (to prevent or ameliorate the effects of a future infection by a pathogen), or therapeutic (to ameliorate a disease that has already occurred, such as cancer). There are several types of vaccines known and used in the art, including, for example, inactivated virus vaccines, live-attenuated virus vaccines, messenger RNA (mRNA) vaccines, subunit vaccines, recombinant vaccines, polysaccharide vaccines, conjugate vaccines, toxoid vaccines, and viral vector vaccines. The administration of vaccines is referred to as “vaccination.”

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Provided herein are vaccine compositions and uses thereof. In particular, provided herein are synthetic viral-like structures (sVLSs) based vaccines and the use of such vaccines to prevent infection by a pathogen (e.g., viral pathogen).

The sVLSs described herein allow for optimization of immunogenicity that is not available in existing vaccine platforms. In addition, the sVLSs are stable and provide easy access to people around the world in contrast to the need of ultracold freezing conditions.

For example, in some embodiments, provided herein is synthetic viral-like structure (sVLS), comprising: a liposome comprising a polypeptide antigen covalently conjugated to the surface of the liposome via a maleimide group of a lipid in the liposome and a thiol group of the polypeptide antigen. In some embodiments, the thiol group is on a cysteine of the polypeptide antigen (e.g., via site-specifically engineering or naturally found in the polypeptide antigen). In some embodiments, the cysteine is selectively reduced (e.g., via tris(2-carboxyethyl) phosphine (TCEP)).

The sVLSs allow for tuning of the density of polypeptide antigen (e.g., via concentration of lipid or polypeptide) to tune immunogenicity (e.g., for different subjects). The present disclosure is not limited to a particular density of polypeptide antigen. In some embodiments, each liposome comprises at least 10, 15, 20, 25, 30, 35, 40, 45, or 50 molecules of the polypeptide antigen.

The present disclosure is not limited to particular lipids. In some embodiments, the lipid is 1,2-distearoyl-sn-glycero-3-phosphocholine (DSPC), 1,2-diheptadecanoyl-sn-glycero-3-phosphocholine, or 1,2-dinonadecanoyl-sn-glycero-3-phosphocholine.

In some embodiments, the liposome encapsulates a nucleic acid (e.g., DNA or RNA) adjuvant (e.g., comprising an all-natural phosphodiester backbone). The present disclosure is not limited to a particular nucleic acid adjuvant. Examples include but are not limited to, 5′-TCCATGACGTTCCTGACGTT-3′ (SEQ ID NO: 1), TCCATGAGCTTCCTGAGCTT-3′ (SEQ ID NO: 2), or 5′-ACUGUUGAUUCAUCACAGGG-3′ (SEQ ID NO: 3), an RNA oligonucleotide comprising uracil, or a DNA oligonucleotide of a random sequence.

A composition of the present disclosure desirably is a pharmaceutically acceptable (e.g., physiologically acceptable) composition, which comprises a carrier, preferably a pharmaceutically acceptable (e.g., physiologically acceptable) carrier and the sVLS described herein. Compositions of the present disclosure may be formulated into pharmaceutical compositions that are administered in a therapeutically effective amount to a subject and may further comprise suitable, pharmaceutically-acceptable excipients, additives, or preservatives. Suitable excipients, additives, and preservatives are well known in the art.

The compositions described herein desirably comprise therapeutically effective amounts of the sVLS. A “therapeutically effective amount” refers to an amount effective, at dosages and for periods of time necessary, to achieve a desired therapeutic result. Alternatively, the pharmacologic and/or physiologic effect may be prophylactic, i.e., the effect completely or partially prevents a disease or symptom thereof. In this respect, the disclosed compositions comprise “prophylactically effective amounts” of the sVLS. A “prophylactically effective amount” refers to an amount effective, at dosages and for periods of time necessary, to achieve a desired prophylactic result (e.g., prevention of subsequent infection and/or disease onset).

Exemplary dosage forms for pharmaceutical administration are described herein, and include, but are not limited to liquids, ointments, creams, emulsions, lotions, gels, bioadhesive gels, sprays, aerosols, pastes, foams, sunscreens, capsules, microcapsules, suspensions, pessary, powder, semi-solid dosage forms, etc. The compositions can be generated in accordance with conventional techniques described in, e.g., Remington: The Science and Practice of Pharmacy, 23rd Edition, Academic Press (2020).

The disclosed compositions can be provided in many different types of containers and delivery systems. For example, in some embodiments, the composition can be presented in unit-dose or multi-dose sealed containers, such as ampules and vials, and can be stored in a freeze-dried (lyophilized) condition requiring only the addition of the sterile liquid carrier, for example, water, immediately prior to use. In some embodiments, the compositions are provided in a suspension or liquid form. Such compositions can be delivered in any suitable container including spray bottles and any suitable pressurized spray device. Such spray bottles may be suitable for delivering the compositions intranasally or via inhalation. These containers can further be packaged with instructions for use to form kits (described below).

The disclosure also provides methods of using the above-described compositions to induce an immune response against an antigen in a subject. The present disclosure is not limited to a particular antigen. In some embodiments, the antigen is a pathogen (e.g., viral) antigen. In some specific embodiments, the polypeptide antigen is SARS-COV-2 receptor binding domain (RBD).

The vaccines described herein may also direct an immune response against cancer cells and can include tumor cell derived antigens, epitopes, and/or neoepitopes, or portions thereof, or nucleic acids encoding tumor cell derived antigens, epitopes, and/or neoepitopes. Tumor antigens are surface molecules that are differentially expressed in tumor cells relative to non-tumor tissues. Tumor antigens make tumor cells immunologically distinct from normal cells and provide diagnostic and therapeutic targets for human cancers. Tumor antigens have been characterized either as membrane proteins or as altered carbohydrate molecules of glycoproteins or glycolipids on the cell surface. Cancer cells often have distinctive tumor antigens on their surfaces, such as truncated epidermal growth factor, folate binding protein, epithelial mucins, melanoferrin, carcinoembryonic antigen, prostate-specific membrane antigen, HER2-neu, which are candidates for use in therapeutic cancer vaccines. Because tumor antigens are normal or related to normal components of the body, the immune system often fails to mount an effective immune response against those antigens to destroy the tumor cells. Illustrative cancer types for which this approach can be used include prostate, colon, breast, ovarian, pancreatic, brain, head and neck, melanoma, leukemia, lymphoma, etc.

In other embodiments, the antigen present in the vaccine composition is not a foreign antigen, but a self-antigen, e.g., the vaccine composition is directed toward an autoimmune disease. Examples of autoimmune diseases include type 1 diabetes, conventional organ specific autoimmunity, neurological disease, rheumatic diseases/connective tissue disease, autoimmune cytopenias, and related autoimmune diseases. Such conventional organ specific autoimmunity may include thyroiditis (Graves+Hashimoto's), gastritis, adrenalitis (Addison's), ovaritis, primary biliary cirrhosis, myasthenia gravis, gonadal failure, hypoparathyroidism, alopecia, malabsorption syndrome, pernicious anemia, hepatitis, anti-receptor antibody diseases and vitiligo. Such neurological diseases may include schizophrenia, Alzheimer's disease, depression, hypopituitarism, diabetes insipidus, sicca syndrome and multiple sclerosis. Such rheumatic diseases/connective tissue diseases may include rheumatoid arthritis, systemic lupus erythematous (SLE) or Lupus, scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris, Sjogren's syndrome. Other autoimmune related diseases may include autoimmune uvoretinitis, glomerulonephritis, post myocardial infarction cardiotomy syndrome, pulmonary hemosiderosis, amyloidosis, sarcoidosis, aphthous stomatitis, and other immune related diseases, as presented herein and known in the related arts.

In some aspects, the disclosure provides use of any of the above-described immunogenic compositions in the preparation of a medicament, such as a medicament for immunizing an animal against a pathogen. In other aspects, the disclosure provides a method of inducing an immune response in a subject, the method comprising administering a therapeutically effective amount of the composition.

In one aspect, the disclosure relates to a method for vaccination against, or for prophylaxis or therapy (prevention or treatment) of exposure to, or infection with, a pathogen (such as those described herein) via administration of a therapeutically or prophylactically effective amount of the compositions described herein to a subject in need thereof. Accordingly, administration of the composition primes, enables, and/or enhances induction of both humoral (e.g., development of specific antibodies) and cellular (e.g., cytotoxic T lymphocyte) immune responses. Cytokines play a role in directing the immune response. Helper (CD4+) T cells orchestrate the immune response of mammals through production of soluble factors that act on other immune system cells, including B and other T cells. Most mature CD4+T helper cells express one of two cytokine profiles: Th1 or Th2. Th1-type CD4+T cells secrete IL-2, IL-3, IFN-γ, GM-CSF and high levels of TNF-α. Th2 cells express IL-3, IL-4, IL-5, IL-6, IL-9, IL-10, IL-13, GM-CSF and low levels of TNF-α. Th1 type cytokines promote both cell-mediated immunity, and humoral immunity that is characterized by immunoglobulin class switching to IgG2a in mice and IgG1 in humans. Th1 responses may also be associated with delayed-type hypersensitivity and autoimmune disease. Th2 type cytokines induce primarily humoral immunity and induce class switching to IgG1 and IgE. The antibody isotypes associated with Th1 responses generally have neutralizing and opsonizing capabilities, whereas those associated with Th2 responses are associated more with allergic responses.

Several factors have been shown to influence skewing of an immune response towards either a Th1 or Th2 type response. The best characterized regulators are cytokines. IL-12 and IFN-γ are positive Th1 and negative Th2 regulators. IL-12 promotes IFN-γ production, and IFN-γ provides positive feedback for IL-12. IL-4 and IL-10 appear important for the establishment of the Th2 cytokine profile and to down-regulate Th1 cytokine production.

For example, in some embodiments, the disclosed method results in the skewing of a host's immune response away from Th2 type immune response and toward a Th1 type immune response. In other words, the disclosed methods may induce a cellular immune response that is a Th1-biased immune response. In some embodiments, the present disclosure provides immunogenic compositions and methods for skewing and/or redirecting a host's immune response (e.g., away from Th2 type immune responses and toward Th1 type immune responses) to one or a plurality of immunogens/antigens. In some embodiments, skewing and/or redirecting a host's immune response (e.g., away from Th2 type immune responses and toward Th1 type immune responses) to one or a plurality of immunogens/antigens comprises providing one or more antigens (e.g., recombinant antigens, isolated and/or purified antigens, antigen-encoding nucleic acid sequences, and/or killed whole pathogens) that are historically associated with generation of a Th2 type immune response when administered to a subject.

With respect to viral infections, “humoral immunity” occurs when virus and/or virus-infected cells stimulate B lymphocytes to produce antibody that is specific for viral antigen. IgG, IgM, and IgA antibodies have all been shown to exert antiviral activity. Such neutralizing antibodies can exert antiviral activity by (1) blocking virus-host cell interactions or (2) recognizing viral antigens on virus-infected cells which can lead to antibody-dependent cytotoxic cells (ADCC) or complement-mediated lysis. IgG antibodies are responsible for most antiviral activity in serum, while IgA is the most important antibody when viruses infect mucosal surfaces.

In some embodiments, the disclosed method reduces the number of booster injections (e.g., of an antigen containing composition) required to achieve a desired immune response (e.g., a protective immune response (e.g., a memory immune response)). In some embodiments, the disclosed method results in a higher proportion of recipients achieving seroconversion and/or more consistent immune responses within a population of subjects administered the immunogenic composition. In some embodiments, the present disclosure provides compositions that are useful for selectively skewing adaptive immunity toward Th1, Th2, or cytotoxic T cell responses (e.g., allowing effective immunization by distinct routes (e.g., such as via mucosa or via injection)). In some embodiments, the present disclosure provides compositions that elicit optimal responses in subjects in which most contemporary vaccination strategies are not optimally effective (e.g., in very young and/or very old populations). Ideally, the disclosed method induces a protective immune response, that is, an immune response that prevents the subject from displaying signs or symptoms of infection upon subsequent exposure of the subject to the pathogen.

In some embodiments, the present disclosure provides compositions that provide efficacy and safety needed for vaccination regimens that involve different delivery routes and elicitation of distinct types of immunity. In some embodiments, the present disclosure provides immunogenic compositions that stimulate antibody responses and have little toxicity and that can be utilized with a range of antigens for which they provide adjuvanticity and the types of immune responses they elicit. In some embodiments, the present disclosure provides immunogenic compositions that meet global supply requirements (e.g., in response to a pandemic such as a coronavirus pandemic).

The compositions of the present disclosure can be administered by any suitable route of administration. It will also be appreciated that the chosen route will vary with the condition and age of the recipient, and the disease being treated.

For example, the compositions can be administered by oral, parenteral (e.g., intramuscular, intraperitoneal, intravenous, ICV, intracisternal injection or infusion, subcutaneous injection, or implant), by nasal, vaginal, rectal, sublingual, urethral (e.g., urethral suppository) or topical routes of administration (e.g., gel, ointment, cream, aerosol, etc.), and can be formulated, alone or together, in suitable dosage unit formulations containing conventional non-toxic pharmaceutically acceptable carriers, adjuvants, excipients, and vehicles appropriate for each route of administration. Non-limiting examples of carriers include phosphate buffered saline (PBS), saline or a biocompatible matrix material such as a decellularized liver matrix (DCM as disclosed in Wang et al. (2014) J. Biomed. Mater Res. A. 102 (4): 1017-1025) for topical or local administration. The compositions can optionally contain a protease inhibitor, glycerol and/or dimethyl sulfoxide (DMSO).

In some embodiments, compositions of the present disclosure are administered mucosally (e.g., using standard techniques; see, e.g., Remington: The Science and Practice of Pharmacy, 23rd Edition, Academic Press (2020) (e.g., for mucosal delivery techniques, including intranasal, pulmonary, vaginal and rectal techniques), as well as European Publication No. 517,565 and Illum et al., J. Controlled Rel., 1994, 29:133-141 (e.g., for techniques of intranasal administration)). Alternatively, the compositions of the present disclosure may be administered dermally or transdermally, using standard techniques (see, e.g., Remington: The Science and Practice of Pharmacy, 23rd Edition, Academic Press (2020)). The present disclosure is not limited by the route of administration.

In some embodiments, the disclosed method is used to protect and/or treat a subject susceptible to, or suffering from, a disease by means of administering the disclosed composition via injection (e.g., via intradermal, subcutaneous, intramuscular, intravenous, intraosseous, intraperitoneal, intrathecal, epidural, intracardiac, intraarticular, intracavernous, and/or intravitreal route). Methods of systemic administration include conventional syringes and needles, or devices designed for ballistic delivery (see, e.g., WO 99/27961), or needleless pressure liquid jet device (see, e.g., U.S. Pat. Nos. 4,596,556 and 5,993,412), or transdermal patches (see, e.g., WO 97/48440 and WO 98/28037). In some embodiments, the present disclosure provides a delivery device for systemic administration, pre-filled with a composition of the present disclosure.

In some embodiments, the composition is administered via a mucosal route (e.g., an oral/alimentary or nasal route). Alternative mucosal routes include intravaginal and intrarectal routes. In some embodiments, a nasal route of administration is used, which is also referred to herein as “intranasal administration” or “intranasal vaccination.” Methods of intranasal vaccination are well known in the art, including the administration of a droplet or spray form of a composition into the nasopharynx of a subject to be immunized. Intranasal administration may also include contact with the oral mucosa, bronchial mucosa, and other epithelia. In some embodiments, a nebulized or aerosolized composition is provided. Formulations for nasal delivery may include those with dextran or cyclodextran and saponin as an adjuvant.

Enteric formulations such as gastro-resistant capsules for oral administration and suppositories for rectal or vaginal administration also may be employed. Compositions of the present disclosure may also be administered via the oral route. Under these circumstances, a composition may comprise a pharmaceutically acceptable excipient and/or include alkaline buffers, or enteric capsules. When the composition is administered via a vaginal route, the composition may comprise pharmaceutically acceptable excipients and/or emulsifiers, polymers (e.g., CARBOPOL), and other known stabilizers of vaginal creams and suppositories. When the composition is administered via a rectal route, the composition may comprise excipients and/or waxes and polymers known in the art for forming rectal suppositories.

Oral compositions can be prepared according to methods known in the art, and such compositions may contain one or more agents selected from the group consisting of sweetening agents, flavoring agents, coloring agents, and preserving agents in order to provide pharmaceutically elegant and palatable preparations. Tablets contain active ingredients in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g., starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in U.S. Pat. Nos. 4,256,108; 4,166,452; and U.S. Pat. No. 4,265,874 to form osmotic therapeutic tablets for control release. The pharmaceutical compositions of the disclosure may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, the same route of administration (e.g., mucosal administration) is chosen for both a priming and boosting vaccination. In other embodiments, multiple routes of administration are utilized (e.g., at the same time, or, alternatively, sequentially) in order to stimulate an immune response (e.g., using one or more compositions of the present disclosure).

In some embodiments, the disclosed composition(s) is/are administered to a mucosal surface of a subject in either a priming or boosting vaccination regime. Alternatively, in some embodiments, a composition is administered systemically in a priming and/or boosting vaccination regime. In some embodiments, an immunogenic composition is administered to a subject in a priming vaccination regimen via mucosal administration and a boosting regimen via systemic administration. In some embodiments, an immunogenic composition is administered to a subject in a priming vaccination regimen via systemic administration and a boosting regimen via mucosal administration. Examples of systemic routes of administration include, but are not limited to, a parenteral, intramuscular, intradermal, transdermal, subcutaneous, intraperitoneal or intravenous administration.

In some embodiments, the composition may be applied and/or delivered utilizing electrophoretic delivery/electrophoresis. Further, compositions may be applied by a transdermal delivery system such as a patch or administered by a pressurized or pneumatic device (i.e., “gene gun”). Such methods, which comprise applying an electrical current, are well known in the art.

The compositions described herein may be administered topically. If applied topically, the compositions may be occluded or semi-occluded. Occlusion or semi-occlusion may be performed by overlaying a bandage, polyoleofin film, article of clothing, impermeable barrier, or semi-impermeable barrier to the topical preparation.

The pharmaceutical compositions for administration may be applied in a single administration or in multiple administrations. Indeed, as discussed above, following an initial administration of a composition of the present disclosure (e.g., an initial vaccination), a subject may receive one or more boost administrations (e.g., around 2 weeks, around 3 weeks, around 4 weeks, around 5 weeks, around 6 weeks, around 7 weeks, around 8 weeks, around 10 weeks, around 3 months, around 4 months, around 6 months, around 9 months, around 1 year, around 2 years, around 3 years, around 5 years, around 10 years) subsequent to a first, second, third, fourth, fifth, sixth, seventh, eighth, ninth, tenth, and/or more than tenth administration. Although an understanding of the mechanism is not necessary to practice the present disclosure and the present disclosure is not limited to any particular mechanism of action, in some embodiments, reintroduction of an immunogen in a boost dose enables vigorous systemic immunity in a subject. The boost can be with the same formulation given for the primary immune response, or can be with a different formulation that contains the immunogen. The dosage regimen will also, at least in part, be determined by the need of the subject and be dependent on the judgment of a practitioner.

Dosage units may be proportionately increased or decreased based on several factors including, but not limited to, the weight, age, and health status of the subject. In addition, dosage units may be increased or decreased for subsequent administrations (e.g., boost administrations).

It is contemplated that the compositions and methods of the present disclosure will find use in various settings, including research settings. For example, compositions and methods of the present disclosure also find use in studies of the immune system (e.g., characterization of adaptive immune responses (e.g., protective immune responses (e.g., mucosal or systemic immunity))). Uses of the compositions and methods provided by the present disclosure encompass human and non-human subjects and samples from those subjects, and also encompass research applications using these subjects. Thus, it is not intended that the present disclosure be limited to any particular subject and/or application setting.

In some embodiments, the present disclosure provides a kit comprising a compositions described herein (e.g., sVLS). In some embodiments, the kit further contains a device for administering compositions. The present disclosure is not limited by the type of device included in the kit. In some embodiments, the device is configured for nasal application of a composition of the present disclosure (e.g., a nasal applicator (e.g., a syringe) or nasal inhaler or nasal mister). In some embodiments, a kit comprises the sVLS composition in a concentrated form (e.g., that can be diluted prior to administration to a subject).

In some embodiments, all kit components are present within a single container (e.g., vial or tube). Alternatively, each kit component may be located in a single container (e.g., vial or tube). In other embodiments, one or more kit components are located in a single container (e.g., vial or tube) with other components of the same kit being located in a separate container (e.g., vial or tube). In some embodiments, the kit comprises a buffer. The kit may further comprise instructions for use.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific Examples that follow are only intended for the purposes of illustration, and are not to be construed as limiting in any manner to make compounds of the disclosure by other methods.

### Example 1

### Maleimide Conjugation Chemistry

The primary reason for choosing maleimide thiol chemistry for construction of sVLSs is the stability of the conjugation gained from a covalent bond, in contrast to other noncovalent conjugation chemistry such as metal chelation coordination chemistry. Metal chelation chemistry leads to gradual loss of the conjugated proteins on liposomes in physiologically relevant conditions.¹ In contrast, for different proteins, conjugation achieved via thiol maleimide chemistry on these structures is much more stable under the same environmental conditions. ²⁻⁴ The second important reason to choose maleimide thiol chemistry is that the maleimide-thiol conjugation is less likely to be a regulatory concern because several FDA approved drug products contain maleimide-thiol conjugates ⁵⁻⁷.

The condition for maleimide thiol conjugation is the presence of free thiol groups on the protein or antigen of interest. A thiol group can be conveniently engineered into the protein of interest through introduction of a site-specific cysteine. However, depending on the source of the protein where it is purified, the thiol may not be always available for maleimide conjugation. Proteins purified from the bacteria E. coli usually have engineered cysteines retained in the reduced free thiol format, as was the case for hen egg lysozyme. ³ However, for proteins purified from mammalian cell culture, this may not be the case.

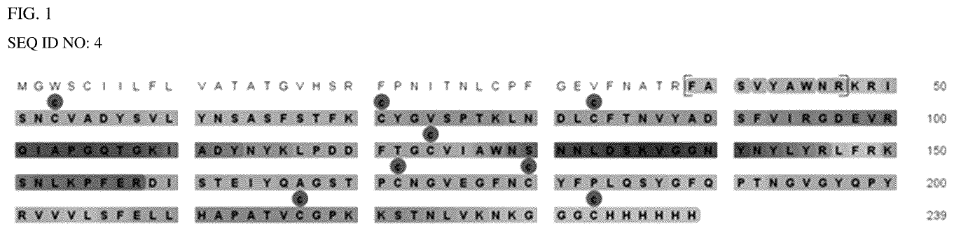

The test antigen was the receptor binding domain (RBD) from the SARS-COV-2 virus, the causative agent of COVID-19. Extensive test in E. coli for expression of RBD itself yields little protein in soluble fractions. In contrast, the human derived 293F system consistently expressed RBD in soluble form and the protein can be secreted into cell culture supernatant for easy harvesting and purification.⁴ However, without additional treatment, this soluble form of RBD is resistant to conjugation using thiol maleimide chemistry. ³ Experiments were performed to examine the underlined cause of this result. First of all, reactive cysteine mapping by mass spectrometry showed that the cysteine that was engineered near the C-terminus of the RBD protein (C233) was in an oxidized form when the protein was purified. As shown below in FIG. 1, this mass spectrometry analysis for the purified RBD identified eight cysteines (highlighted in red) that were oxidized in the purified protein, which includes C233 that was engineered for the purpose of conjugation.

Among these 8 identified cysteines, 7 of them are expected to be oxidized based on the published structures of RBD since they are involved in disulfide bond formation in the native RBD protein. ⁸⁻¹¹. The oxidation of C233 was unexpected but the results from mass spectrometry were very strong as shown by the A-Score in the table below:

TABLE 1
Table 1. The list of cysteines identified by mass spectrometry to
be oxidized, with localization probability and the best A-Score.

| | | Best | Localization | Modified | Total |
|---|---|---|---|---|---|
| | | A- | Probability | SpC | SpC |
| Site | Modification | Score | (%) | RBD2 | RBD2 |
| C53 | Carbamidomethyl | 1,000.00 | 100 | 25 | 25 |
| C71 | Carbamidomethyl | 1,000.00 | 100 | 42 | 42 |
| C83 | Carbamidomethyl | 1,000.00 | 100 | 35 | 35 |
| C124 | Carbamidomethyl | 1,000.00 | 100 | 33 | 33 |
| C172 | Carbamidomethyl | 1,000.00 | 100 | 27 | 27 |
| C180 | Carbamidomethyl | 1,000.00 | 100 | 22 | 22 |
| C217 | Carbamidomethyl | 1,000.00 | 100 | 53 | 53 |
| C233 | Carbamidomethyl | 1,000.00 | 100 | 5 | 5 |

The table above lists the cysteines identified by mass spec that were modified by light isotope iodoacetamide after reduction using 10 mM DTT. The table includes residue and site number, as well as best A-Score, localization probability and the number of spectra supporting the identification of the site. A-Score of 13 are significant and the highest A-Score is 1,000.

### Selective Reduction of Cysteines Using Sub-Stoichiometric Amount of TCEP

In order to conjugate C233 without disruption of all the other existing disulfide bonds in RBD, trace amounts of tris(2-carboxyethyl) phosphine (TCEP) were used for selective reduction of C233 while maintaining all the other disulfide bonds in RBD. The rationale for using trace amount of TCEP is as follows: (1) reduction by TCEP is irreversible and stoichiometric.¹² (2) the physical location of C233 is near the C-terminus of the protein, which is more solvent exposed than all the other cysteines in this protein. Therefore, the major target of TCEP is C233 instead of other cysteines in the RBD protein.

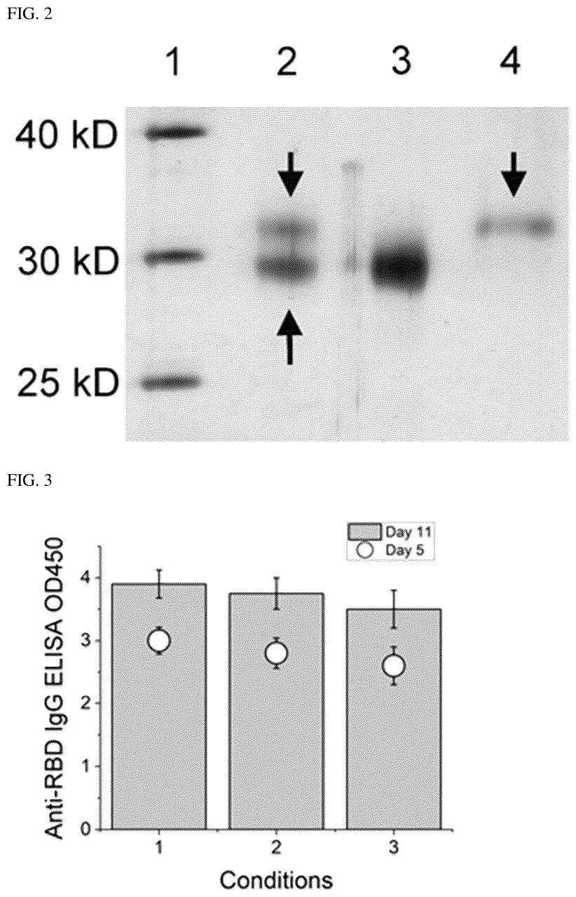

In order for this idea to work, the amount of TCEP added into purified RBD was carefully chosen, because any excess TCEP, if present, will be able to reduce additional oxidized cysteines. To this end, the molar quantity of the engineered thiol (C233) based on concentration of RBD was determined using UV/VIS absorbance method, and the amount of TCEP that can reduce 80% of all the available C233 was used. The reason to choose 80% instead of 100% as a cutoff is to ensure that other cysteines that are involved in disulfide bonds are not reduced. The specific amount of TCEP was added into the RBD protein solution 10 min before addition of liposomes at 20° C. No additional steps to remove TCEP are necessary. As shown in FIG. 2, this selective reduction of C233 worked.⁴

As shown in FIG. 2, the lipid conjugate with C233 is indicated by the downward arrow in lane 2 and lane 4, respectively. The sample for lane 2 is before size exclusion chromatography (SEC), where the free protein still present in the sample is also indicated by the upward arrow. Compared to the free protein, the amount of mobility shift on the gel by the conjugate is fully consistent with the molecular weight of one maleimide-containing lipid, which is 2941.605 Dalton. In order to confirm the site specificity of maleimide reaction, another RBD protein with the C233 near the C-terminus of RBD mutated to alanine (RBD-Ala) was generated. Under the same set of conditions as in lane 2, the purified RBD-Ala did not yield any conjugation between the protein and liposomes, as shown in lane 3. This result indicates that the conjugation observed in lane 2 is specific to the C233 that was engineered near the C-terminus of the RBD. Under these conditions, none of the existing disulfide bonds in RBD were reduced and thus none of those cystines participated in the maleimide reaction. This demonstrates site-specific conjugation of RBD onto liposomes, which results in orientation-specific display of the RBD proteins on the surface of the liposome, similar to that in authentic viruses. By further using SEC, the RBD-conjugated liposomes (pRBD) are purified away from free proteins, as shown in lane 4. This result also indicates that RBD were attached to liposomes through covalent conjugation, and under these conditions, non-specific or noncovalent association between RBD and liposomes is negligible.

Lipids with High Melting Temperature

An important feature of sVLSs is that they can encapsulate nucleic acids in the interior of the structures for modulation of immunogenicity. Thus, nucleic acids with natural backbones are protected from nuclease degradation as a result of this encapsulation. To ensure the stability of this encapsulation, the lipids used in these sVLSs should have a melting temperature that is well above the normal body temperature of 37° C. With a high melting temperature and in the presence of minor quantity of cholesterol, these structures retain nucleic acids (NA) without leakage. For this purpose, 1,2-distearoyl-sn-glycero-3-phosphocholine (DSPC) instead of 1,2-dimyristoyl-sn-glycero-3-phosphocholine (DMPC) was used for making these sVLSs. DSPC has a reported melting temperature of 55° C. ¹³ Thus at physiological temperatures, these lipid molecules remain closely packed in the ordered gel phase, which affords better retention of molecules inside these structures, including RNA molecules. ⁴

Moreover, in sVLSs, DSPC affords one more important advantage over other lipid molecules used for delivery of NA molecules: charge neutrality. At physiological pH 7.3-7.4, the overall charge on DSPC is zero. This neutrality resembles the majority of natural phospholipid molecules in plasma membrane and keeps these structures inert by themselves in the absence of conjugated proteins. In contrast, charged lipids have been shown in many instances to be immunostimulatory and therefore enhance the probability of undesired side reaction or reactogenicity upon injection.

The resulting sVLSs are very stable when stored as a suspension in PBS at 4° C. The efficacy of the sVLS upon storage at 4° C. with time is shown using an sVLS that display on average 46 molecules of RBD on its surface and 139 molecules of DNA1 oligos in its interior. The sequence of DNA1 is as follows: 5′-TCCATGACGTTCCTGACGTT-3′ (SEQ ID NO: 1) with all-natural phosphodiester backbone, which harbors two unmethylated CpG dinucleotides and serves as a ligand for Toll-like receptor 9 (TLR9) ¹⁴. This specific sVLS was synthesized as reported ⁴ and stored at 4° C. On Day 2, 6 months and 1 year after storage at 4° C., the sVLS was taken for subcutaneous (SC) immunization of C57BL6/J mice at a dose of 0.24 μg of RBD per animal. The sera from animals were collected on Day 5 and Day 11 post immunization and assayed for RBD-specific ELISA. Over the course of 1 year, there is only a minor drop (less than 15%) in the efficacy of the sVLS as measured by anti-RBD IgG ELISA (FIG. 3), demonstrating that these structures are very stable upon storage at 4° C.

sVLS with DNA with all-Natural Phosphodiester Backbone

DNA as adjuvants for vaccines is well established both in scientific community and also in pharmaceutical industry. However, to ensure the potency of the DNA adjuvants, most of the existing practices that involve DNA adjuvants use DNA with modified phosphorothioate backbones. The phosphorothioate backbone allows the DNA molecules to resist degradation by nucleases in biological environment and therefore affords longer duration of the efficacy. However, the downside of employing these “unnatural” DNA molecules in vivo, especially for human therapeutic applications is the fate of these DNA molecules in the body, is that these “unnatural” DNA can accumulate inside the tissue and the potential long-term side effects are poorly characterized.

In contrast, the sVLS system uses DNA with completely natural phosphodiester backbone. This is possible because sVLS encapsulate the DNA molecules in the interior of the structures, and therefore protect these molecules from degradation until they reach their target site within the antigen-specific B cells. The advantages of delivering these DNA molecules this way are threefold: (1) the efficacy of the adjuvant effect is fully preserved; (2) these DNA molecules can be degraded by nucleases without the concern of long-term accumulation, which is a much safer than DNA with modified phosphorothioate backbones, and (3) manufacturing cost for DNA with natural backbones is much less compared to DNA with phosphorothioate backbones.

Tuning the Immunogenicity of sVLSs

Compared to various existing vaccine platforms, a major yet unique advantage of sVLSs is the ability to finely tune the immunogenicity of these structures and the ability to optimize these structures in vitro for in vivo applications. This feature is desired given the diversity of human population. For example, people of different age groups have different responses towards the same vaccines due to age dependent changes in the immune system. People who are immunocompromised need to be better protected than normal individuals. In practice this is usually managed by changing the vaccines doses or the number of boosters. However, the range of immune protection offered by changing vaccine dose or the number of boosters is rather limited. The mRNA COVID-19 vaccine offers a perfect example in this regard, where a lower vaccine dose nevertheless produces a lower protection in pediatric patients under 5 years old.

The unique advantage of sVLS is the ability to tune the immunogenicity of the structure using a single platform. Specifically, the immunogenicity of these structures can be tuned in two different ways: (1) by changing the average epitope density displayed on the outer surface of these structures, and (2) by changing the sequence and concentration of the internal nucleic acids.

Specifically, the epitope density can be changed by two variables during the synthesis of these structures: (1) the molar percentage of the maleimide-containing phospholipid molecules used during the formation of these structures, and (2) the concentration of the protein antigen used in the conjugation reaction. Lower percentage of the maleimide-containing lipid or lower concentration of the protein leads to lower epitope density for the antigens of interest and vice versa. The concentration of the internal nucleic acids is changed by changing the initial concentration of the nucleic acids included during the formation of these structures.

Th effect of changing the average epitope density on the immunogenicity of these structures is shown for two different antigens: (1) the receptor binding domain (RBD) of the SARS-COV-2 virus, and (2) a mutant form of hen egg lysozyme (HELD) that carries two site-directed mutations R73E and D101R¹⁵.

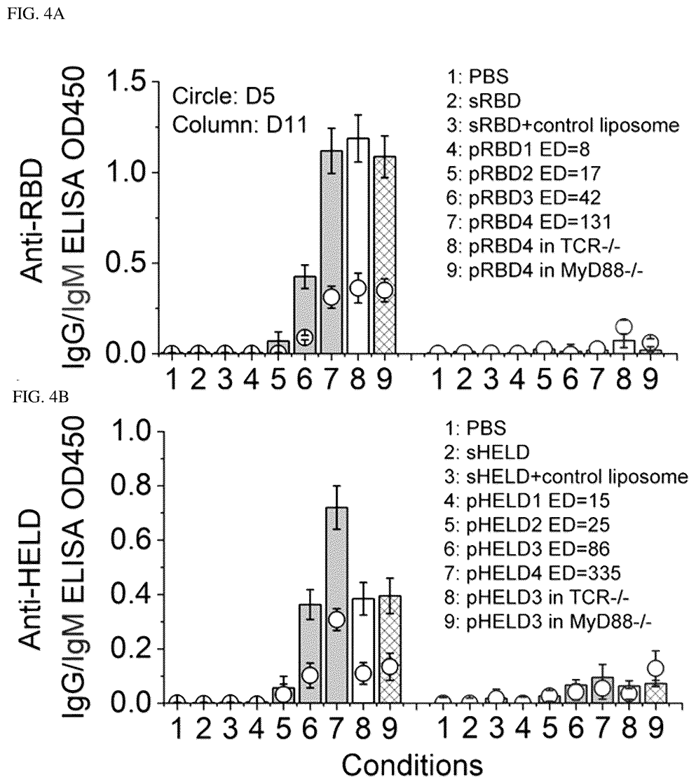

To examine the effect of epitope density on the immunogenicity of these structures, all the above experiments used sVLS with PBS in the interior of these structures to isolate the effect of epitope density. For the 9 different conditions that we have tested above, it is very clear that, for both RBD and HELD, these structures elicit an antigen-specific B cell IgG response provided that the epitope density is above a threshold. This threshold is between 17 and 42 for RBD and 25 and 86 for HELD. Above the respective threshold, the IgG response gets higher with increasing epitope density for both RBD and HELD. RBD and HELD are two unrelated protein antigens. The qualitative similar trend as shown in FIG. 4 a and b indicates that this trend may be of general relevance to different foreign protein antigens. For both RBD and HELD, comparison with immunization results obtained in TCR^{-/-} mice (Condition 8) indicates that the antigen-specific IgG response is independent of T cells, and also independent of Toll-like receptor signaling as indicated by the results obtained from MyD88^{-/-} mice (Condition 9).

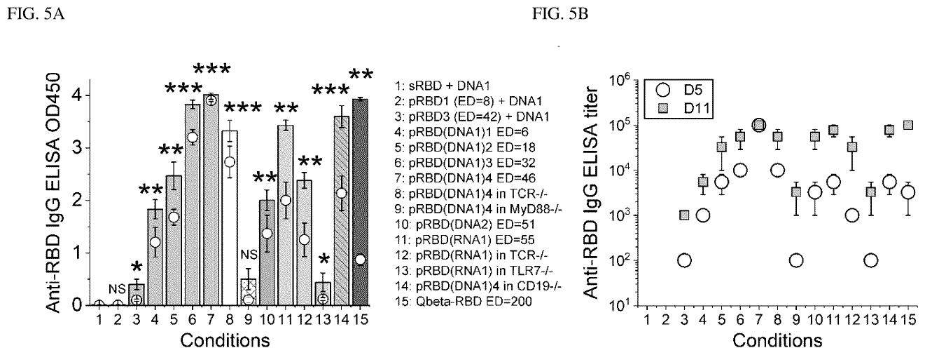

The effect of internal nucleic acids on the immunogenicity of these structures is shown in FIG. 5 for RBD conjugated sVLSs. One can draw several important conclusions based on the results shown in FIG. 5. First of all, an admixture of DNA with RBD conjugated sVLS (pRBD1 and pRBD3, PBS as interior) does not show the adjuvant effect of DNA (Conditions 2 and 3), consistent with the fact that these DNA molecules, when added in trans to the sVLS, are actually degraded in the biological environment.³ In contrast, when DNA1, a 20-mer single-stranded DNA with the following sequence, 5′-TCCATGACGTTCCTGACGTT-3′ (SEQ ID NO: 1) with all natural phosphodiester backbone, was encapsulated in the interior of these structures, a highly potent adjuvant effect was observed (Conditions 4,5,6,7 in wild-type C57BL6/J mice). DNA1 harbors two unmethylated CpG dinucleotides and serves as a ligand for Toll-like receptor 9 (TLR9) 14. This effect is stronger with higher epitope density as one compares across Conditions 4 through 7. This synergistic effect between DNA1 and epitope density is independent of T cells (Condition 8) but does depend on Toll-like receptor signaling (Condition 9), consistent with expectation. Moreover, this adjuvant effect is also observed but weaker when we replaced DNA1 with another 20-mer DNA2 of a different sequence (Condition 10). DNA2 has the following sequence: 5′-TCCATGAGCTTCCTGAGCTT-3′ (SEQ ID NO: 2), which harbored two unmethylated GpC dinucleotides. This result is consistent with the findings that natural DNA can bind to ¹⁶ and activate TLR9 in a sequence-independent manner ¹⁷, but the magnitude of this activation is less for DNA2 compared with DNA1.

Furthermore, the adjuvant effect is observed when RNA1 is encapsulated in the interior of these structures (Condition 11), which is stronger than DNA2 in potency but weaker than DNA1. Specifically, RNA1 is a 20-mer genomic RNA fragment with the following sequence, 5′-ACUGUUGAUUCAUCACAGGG-3′ (SEQ ID NO: 3), which is highly conserved among SARS-COV-1, SARS-COV-2 and MERS-COV, and encodes motif V of an essential RNA helicase in these coronaviruses. This effect of RNA1 does not depend on T cell help (Condition 12) but does depend on Toll-like receptor 7 (Condition 13), consistent with expectations. Note that in FIG. 5, the average number of NA molecules per sVLS are 139 for DNA1, 138 for DNA2, and 147 for RNA1, which are regulated by the concentrations of NA used during formation of sVLS. Given the comparable number of NA molecules per sVLS, these results demonstrate that the immunogenicity of these structures can be tuned by nucleic acid of different sequences that are encapsulated in the interior of these structures. Lastly, sVLS with DNA1 encapsulated in these structures can even elicit potent RBD-specific IgG in mice that are deficient in CD19 coreceptors (Condition 14). This result is highly significant because CD19 is known to lower the threshold for antigen receptor stimulation in B cells ¹⁸ and human patients who carry homozygous mutations in CD19 responded poorly to vaccination.¹⁹ These results indicate that these sVLSs can overcome the immunodeficiency caused by CD19 mutations and therefore benefit this special group of immunocompromised patients.

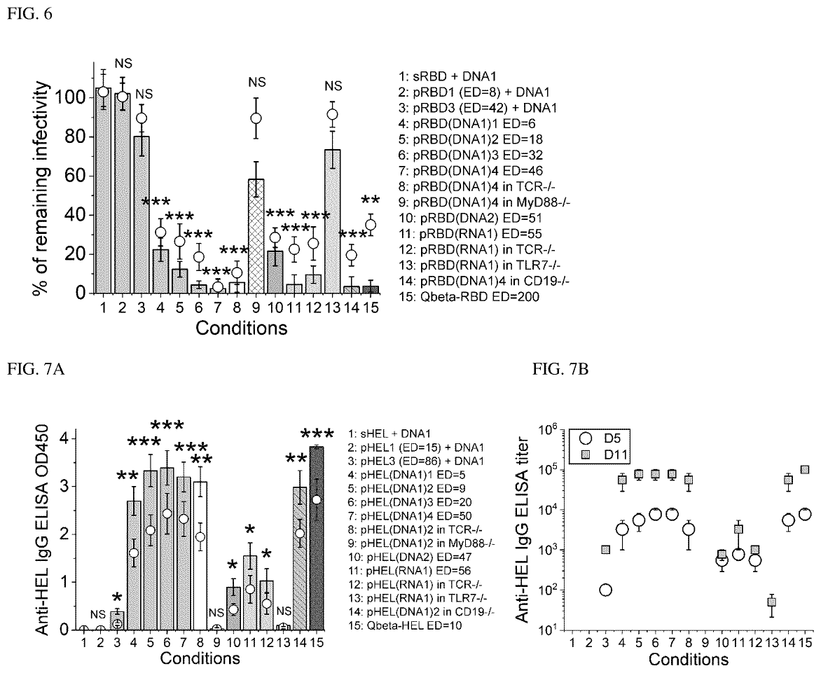

The IgG secreted by the animals in response to the various RBD-conjugated sVLS is protective for the animals, as shown by the neutralization assay summarized in FIG. 6. As shown in FIG. 6, for epitope density as low as 6, the strong IgG induced by pRBD(iNA) as early as D5 after injection can potently neutralize HIV-1 pseudovirions that display the cognate S protein of SARS-COV-2 in vitro, with an even stronger potency on D11 after immunization. These strong neutralizing activities were obtained for all iNA sequences that were (Conditions 4-7, Condition 10 and Condition 11), with condition 7 being the most potent, showing a broad relevance of these results for vaccine development. Even in the absence of T cells, this neutralizing antibody response was strong enough to potently neutralize cognate virions in vitro (Conditions 8 and 12 in FIG. 6). These results are quantitatively consistent with the ELISA data shown in FIG. 5, where DNA1 can induce the most potent RBD-specific IgG response as compared to DNA2 or RNA1. This fast and potent neutralizing antibody response was also mounted in CD19^{-/-} mice (Condition 14 in FIG. 6), confirming that the coreceptor CD19 is not required for a potent nAb response, and that the integrative signals from epitope density and internal nucleic acids on SVLS are strong enough to overcome the apparent deficiency in CD19.

Quantitatively similar to RBD, strong IgG responses specific to HELD was observed upon single SC injections of sVLS conjugated with HELD in the presence of various internal nucleic acids (FIG. 7). As shown in FIG. 7a, these HELD-specific IgG responses arose as early as Day 5 after immunization in wild-type C57BL/6J mice (circles, Conditions 4 through 7, 0.1 μg HELD, mass of protein on sVLS throughout), and continued to rise on Day 11.

To determine the mode of action for the internal DNA, a side-by-side control, in which pHELD3 was mixed with DNA1 in soluble state (pHELD3+DNA1, Condition 3), and this admixture was injected SC into C57BL/6J mice was performed. For this control, the doses of HELD and DNA1 were kept exactly the same as those in pHELD(DNA1). This immunization yielded a 100× lower IgG titer than pHELD(DNA1) on Day 11 (FIG. 7b). This comparison indicates that the internal DNA acts intrinsically and synergistically on antigen-specific B cells to boost IgG responses to antigen on sVLS surface. This lack of action by external DNA was likely due to its quick degradation in biological milieu, similar to Conditions 2 and 3 in FIG. 5 above for RBD. These strong IgG responses revealed potent adjuvant effect of DNA when it was presented in a form similar to that in a virus. These strong IgG responses were independent of T cells (Conditions 8 and 12 in FIG. 7), indicating that these fast IgG responses bypass the typical requirement for T-cell help and drive effective B cell responses in vivo, qualitatively consistent with the observations for sVLS conjugated with RBD (FIG. 5). Although enhancement of IgG by TLR ligands has been reported in numerous instances ²⁰⁻²³, the result shown here distinguishes itself from all previous studies in its enormous and unprecedented synergy (up to 1,000-fold enhancement in titer), low antigen dose and T-independence. The emergence of these rapid, potent and class-switched IgG indicates a novel mechanism of host immune protection against viral infection before T-cell help becomes available. A mechanism of this kind is relevant for viral infection because it was observed that influenza-specific IgG could be mounted in influenza-infected mice that were defective in cognate T cell help ²⁴, and this IgG promoted resolution of primary influenza virus infection and also prevented reinfection in mice ²⁴.

The Ab responses induced by SVLS with internal nucleic acids (iNA) (SVLS (iNA) herein) were compared to a commonly used bacteriophage VLP-based vaccine platform ^{25,26}. The strong IgG induced by SVLS (iNA) was comparable in titers to those elicited by RBD or HEL conjugated on bacteriophage QB VLPs at the same Ag dose, despite the complete absence of the bacteriophage QB coat proteins in these SVLS (iNA). This was shown by Condition 15 in FIG. 5 for a QB-RBD conjugate with an ED of 200 and Condition 15 in FIG. 7 for a Qβ-HEL conjugate with an ED of 10 in wtB6. One-way ANOVA tests on these titer values revealed that Qβ-RBD induced the same level of RBD-specific IgG titers on D5 as pRBD(DNA1) 1 and pRBD(DNA1) 2, which were lower than those for pRBD(DNA1) 3 and pRBD(DNA1) 4; and Qβ-RBD induced the same level of RBD-specific IgG titers on D11 as pRBD(DNA1) 3 and pRBD(DNA1) 4, which were higher than those for pRBD(DNA1) 1 and pRBD(DNA1) 2. For HEL, Qβ-HEL induced the same level of HEL-specific IgG titers on D5 and D11 as all pHEL(DNA1). Bacteriophage VLPs are known to display superior immunogenicity ^{25,26}. These results demonstrate that it is possible to elicit IgG responses of a similar magnitude to bacteriophage VLPs using highly purified components.

The results in FIG. 5 and FIG. 7 show that sVLSs are very potent in elicitation of neutralizing antibody responses, especially when nucleic acids are encapsulated inside these structures. In fact, the neutralizing antibody responses induced by these structures are also durable, which is a much-needed attribute for the next-generation vaccines.

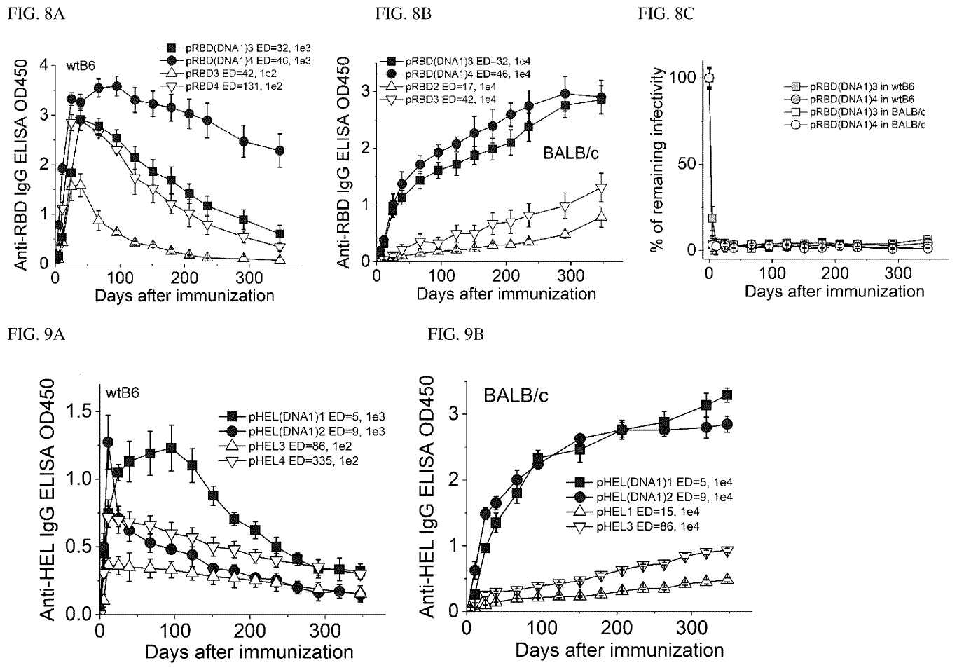

As shown in FIG. 8a, the RBD-specific IgG in wtB6 mice elicited by pRBD(DNA1) peaked around 2 months after the exposure and then slowly decayed over the time span of one year (blue solid symbols). Without internal DNA1, the IgG response was at least 10-fold lower in magnitude (note the different sera dilution factors listed in figure inset), peaked around one month after exposure, and decayed almost to background level over the course of one year. In contrast, the same agents induced long-lasting IgG whose magnitudes increased with time in BALB/c mice, as shown in FIG. 8b. The growing trends were true for both pRBD and pRBD(DNA1), but the presence of DNA1 elicited much higher IgG levels. Despite the waning level of the RBD-specific IgG in wtB6 over time, sera collected over the one-year period remained highly effective in neutralization of cognate pseudovirions (grey symbols in FIG. 8c). Sera from immunized BALB/c mice collected over a year after immunization also had strong neutralizing activity (white symbols in FIG. 8c). All these neutralization activities came from just a single injection of pRBD(DNA1) at a submicrogram dose in the absence of any other adjuvants.

The trends of Ag-specific IgG that varied with host animals were also observed for HEL-conjugated SVLS, as shown in FIG. 9a for wtB6 upon a single injection of pHEL(DNA1) or pHEL. For all cases in B6, the levels of HEL-specific IgG waned over time. The peak values for HEL-specific IgG induced by pHEL(DNA1) (blue symbols in FIG. 9a) were much lower than those for RBD-specific IgG induced by pRBD(DNA1) (blue symbols in FIG. 8a), likely due to the absence of T cell help for HEL in wtB6. In contrast, both pHEL and pHEL(DNA1) induced long-lasting HEL-specific IgG that grew with time in magnitudes in BALB/c mice (FIG. 9b), quantitatively similar to those trends for pRBD and pRBD(DNA1) in BALB/c mice (FIG. 8b). Also, with internal DNA1, the HEL-specific IgG was much stronger than those induced by pHEL without iNA over the one-year period, analogous to the situation induced by pRBD(DNA1) in comparison to pRBD in BALB/c mice over the same period of time. The presence of iNA induced a stronger and more durable neutralizing antibody response compared to SVLS without iNA. The durable IgG response from pHEL(DNA1) in B6 mice (FIG. 9a blue symbols) indicates that long-lived plasma cells may have been generated in the absence of cognate T cell help. A single injection at a dose of 0.1 μg of HELD yielded a strong and potent anti-HELD IgG response in these animals that lasted for more than one year in time (blue symbols in FIG. 9b), and notably, the antibody response gets stronger with time instead of waning with time.

From FIG. 8 and FIG. 9, it is clear that sVLS can induce long-term antibody immunity for both HELD and RBD, and in addition, epitope density and internal nucleic acids are important variables that will modulate the quantitative feature of long-term IgG response.

### References

1. Chen Z, Moon J J, Cheng W. Quantitation and Stability of Protein Conjugation on Liposomes for Controlled Density of Surface Epitopes. Bioconjug Chem. 2018; 29 (4): 1251-1260.
2. Chen Z, Wholey W Y, Hassani Najafabadi A, et al. Self-Antigens Displayed on Liposomal Nanoparticles above a Threshold of Epitope Density Elicit Class-Switched Autoreactive Antibodies Independent of T Cell Help. J Immunol. 2020; 204 (2): 335-347.
3. Wholey W Y, Mueller J L, Tan C, Brooks J F, Zikherman J, Cheng W. Synthetic

Liposomal Mimics of Biological Viruses for the Study of Immune Responses to Infection and Vaccination. Bioconjug Chem. 2020; 31 (3): 685-697.
4. Wholey W Y, Yoda S T, Cheng W. Site-Specific and Stable Conjugation of the SARS-CoV-2 Receptor-Binding Domain to Liposomes in the Absence of Any Other Adjuvants Elicits Potent Neutralizing Antibodies in BALB/c Mice. Bioconjug Chem. 2021; 32 (12): 2497-2506.
5. Senter P D, Sievers E L. The discovery and development of brentuximab vedotin for use in relapsed Hodgkin lymphoma and systemic anaplastic large cell lymphoma. Nat Biotechnol. 2012; 30 (7): 631-637.
6. Peddi P F, Hurvitz S A. Trastuzumab emtansine: the first targeted chemotherapy for treatment of breast cancer. Future Oncol. 2013; 9 (3): 319-326.
7. Lang L. FDA approves Cimzia to treat Crohn's disease. Gastroenterology. 2008; 134 (7): 1819.
8. Lan J, Ge J, Yu J, et al. Structure of the SARS-COV-2 spike receptor-binding domain bound to the ACE2 receptor. Nature. 2020.
9. Wang Q, Zhang Y, Wu L, et al. Structural and Functional Basis of SARS-COV-2 Entry by Using Human ACE2. Cell. 2020.
10. Shang J, Ye G, Shi K, et al. Structural basis of receptor recognition by SARS-COV-2. Nature. 2020.
11. Yan R, Zhang Y, Li Y, Xia L, Guo Y, Zhou Q. Structural basis for the recognition of SARS-COV-2 by full-length human ACE2. Science. 2020; 367 (6485): 1444-1448.
12. Han J C, Han G Y. A procedure for quantitative determination of tris(2-carboxyethyl)phosphine, an odorless reducing agent more stable and effective than dithiothreitol. Anal Biochem. 1994; 220 (1): 5-10.
13. Mabrey S, Sturtevant J M. Investigation of phase transitions of lipids and lipid mixtures by sensitivity differential scanning calorimetry. Proc Natl Acad Sci USA. 1976; 73 (11): 3862-3866.
14. Hemmi H, Takeuchi O, Kawai T, et al. A Toll-like receptor recognizes bacterial DNA. Nature. 2000; 408 (6813): 740-745.
15. Paus D, Phan T G, Chan T D, Gardam S, Basten A, Brink R. Antigen recognition strength regulates the choice between extrafollicular plasma cell and germinal center B cell differentiation. J Exp Med. 2006; 203 (4): 1081-1091.
16. Li Y, Berke I C, Modis Y. DNA binding to proteolytically activated TLR9 is sequence-independent and enhanced by DNA curvature. EMBO J. 2012; 31 (4): 919-931.
17. Haas T, Metzger J, Schmitz F, et al. The DNA sugar backbone 2′ deoxyribose determines toll-like receptor 9 activation. Immunity. 2008; 28 (3): 315-323.
18. Carter R H, Fearon D T. CD19: lowering the threshold for antigen receptor stimulation of B lymphocytes. Science. 1992; 256 (5053): 105-107.
19. van Zelm M C, Reisli I, van der Burg M, et al. An antibody-deficiency syndrome due to mutations in the CD19 gene. N Engl J Med. 2006; 354 (18): 1901-1912.
20. Hou B, Saudan P, Ott G, et al. Selective utilization of Toll-like receptor and MyD88 signaling in B cells for enhancement of the antiviral germinal center response. Immunity. 2011; 34 (3): 375-384.
21. Eckl-Dorna J, Batista F D. BCR-mediated uptake of antigen linked to TLR9 ligand stimulates B-cell proliferation and antigen-specific plasma cell formation. Blood. 2009; 113 (17): 3969-3977.
22. Rookhuizen D C, DeFranco A L. Toll-like receptor 9 signaling acts on multiple elements of the germinal center to enhance antibody responses. Proc Natl Acad Sci USA. 2014; 111 (31): E3224-3233.
23. Akkaya M, Akkaya B, Sheehan P W, et al. T cell-dependent antigen adjuvanted with DOTAP-CpG-B but not DOTAP-CpG-A induces robust germinal center responses and high affinity antibodies in mice. Eur J Immunol. 2017; 47 (11): 1890-1899.
24. Lee B O, Rangel-Moreno J, Moyron-Quiroz J E, et al. CD4 T cell-independent antibody response promotes resolution of primary influenza infection and helps to prevent reinfection. J Immunol. 2005; 175 (9): 5827-5838.
25. Bachmann M F, Jennings G T. Vaccine delivery: a matter of size, geometry, kinetics and molecular patterns. Nat Rev Immunol. 2010; 10 (11): 787-796.
26. Chackerian B, Lowy D R, Schiller J T. Conjugation of a self-antigen to papillomavirus-like particles allows for efficient induction of protective autoantibodies. J Clin Invest. 2001; 108 (3): 415-423.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Although the foregoing subject matter has been described in some detail by way of illustration and example for purposes of clarity of understanding, it will be understood by those skilled in the art that certain changes and modifications can be practiced within.

That which is claimed:

1. A synthetic viral-like structure (sVLS), comprising:
A liposome comprising a polypeptide antigen covalently conjugated to the surface of said liposome via a maleimide group of a lipid in said liposome and a thiol group of said polypeptide antigen.

2. The sVLS of claim 1, wherein said thiol group is on a cysteine.

3. The sVLS of claim 2, wherein said cysteine is site-specifically engineered onto said polypeptide antigen.

4. The sVLS of claim 2, wherein said cysteine is naturally found in said polypeptide antigen.

5. The sVLS of any one of claims 2 to 4, wherein said cysteine is selectively reduced.

6. The sVLS of claim 5, wherein said selectively reduced is via tris(2-carboxyethyl) phosphine (TCEP).

7. The sVLS of any of the preceding claims, wherein each liposome comprises at least 5 molecules of said polypeptide antigen.

8. The sVLS of any of the preceding claims, wherein each liposome comprises at least 20 molecules of said polypeptide antigen.

9. The sVLS of any of the preceding claims, wherein said lipid is selected from the group consisting of 1,2-distearoyl-sn-glycero-3-phosphocholine (DSPC), 1,2-distearoyl-sn-glycero-3-phosphoethanolamine-N-[maleimide (polyethylene glycol)-2000], 1,2-diheptadecanoyl-sn-glycero-3-phosphocholine, and 1,2-dinonadecanoyl-sn-glycero-3-phosphocholine.

10. The sVLS of any of the preceding claims, wherein said liposome encapsulates a DNA or RNA adjuvant.

11. The sVLS of claim 10, wherein said DNA or RNA adjuvant comprises an all-natural phosphodiester backbone.

12. The sVLS of claim 10 or 11, wherein said DNA or RNA adjuvant is selected from the group consisting of 5′-TCCATGACGTTCCTGACGTT-3′, TCCATGAGCTTCCTGAGCTT-3′, and 5′-ACUGUUGAUUCAUCACAGGG-3′.

13. The sVLS of any of the preceding claims, wherein said polypeptide antigen is SARS-CoV-2 receptor binding domain (RBD).

14. A composition, kit, or system comprising the sVLS of any of the preceding claims.

15. The composition, kit, or system of claim 14, wherein said composition is a pharmaceutical composition.

16. The composition, kit, or system of claim 15, further comprising a pharmaceutically acceptable carrier.

17. The composition, kit, or system of any one of claims 14 to 16, further comprising a delivery device.

18. A method of generating an immune response to an antigen, comprising, administering the composition of any one of claims 14 to 16 to a subject in need thereof.

19. The method of claim 18, wherein said antigen is a viral antigen.

20. The method of claim 19, wherein said viral antigen is SARS-COV-2 RBD.

21. A method of preventing a viral infection, comprising, administering the composition of any one of claims 14 to 16 to a subject in need thereof.

22. The use of the composition of any one of claims 14 to 16 to generate an immune response to an antigen in a subject.

23. The use of the composition of any one of claims 14 to 16 to prevent a viral infection in a subject.
